(19) United States
(12) Patent Application Publication
(10) Pub. No.: US 2016/0220184 A1
(43) Pub. Date: Aug. 4, 2016

(54) HYDRATION MONITOR

(71) Applicant: Empire Technology Development LLC, Wilmington, DE (US)

(72) Inventor: Michael Keoni Manion, Seattle, WA (US)

(21) Appl. No.: 14/610,656

(22) Filed: Jan. 30, 2015

(51) Int. Cl. A61B 5/00; G01F 22/00

(52) U.S. Cl. CPC A61B 5/4875; A61B 5/443; A61B 5/7278; A61B 5/746; A61B 5/6813; A61B 5/4266; G01F 22/00

(57) ABSTRACT

Techniques for hydration monitoring are discussed herein. An example technique may include receiving a fluid intake value from a flow meter. The flow meter may be disposed on a beverage container and the fluid intake value may be indicative of an amount of fluid dispensed to an individual. The example technique may further include receiving a fluid output from a fluid excretion module. The fluid output value may be indicative of an amount of fluid excreted by the individual. The example technique may further include determining a hydration status of the individual based on a combination of the fluid intake value and the fluid output value. The hydration status may then be provided to the user.

### BACKGROUND

Determining the hydration status of an individual in real-time may be difficult. If dehydration occurs, significant physical and/or mental impairment may occur. Similarly, over-hydrating may affect an individual's health. It may be important to maintain sufficient hydration of tissues to afford enhanced or sufficient performance or health in various scenarios, such as extreme physical exertion and in medical treatment. Hydration maintenance may also be critical for working and sports animals such as rescue dogs, thoroughbreds, and in the veterinary world. Hydration may conventionally be regarded as the balance of fluid entering an individual and the fluid leaving the individual. In some cases, for example, the amount of fluid leaving an individual by perspiration may be significant, such as during physical exercise. In the medical setting, fluid loss may be due to emesis, for example. It may be important, then, to ensure hydration is maintained by individuals and animals to maintain optimal physical and mental health.

### SUMMARY

Techniques are generally described that include methods and systems for monitoring the hydration status of a user. In some examples, a method may include receiving a fluid intake value from a flow meter disposed on a beverage container. The fluid intake value may be indicative of an amount of fluid dispensed to an individual. In some examples, a method may comprise receiving a fluid output value from a hydration monitor. The fluid output value may be indicative of an amount of water excreted by the individual. In some examples, a method may further comprise determining a hydration status based on a combination of the fluid intake value and the fluid output value.

An example system includes a fluid dispensing unit, a fluid excretion module, and a hydration monitoring unit. The fluid dispensing unit may be configured to provide fluid intake data, wherein the fluid intake data is indicative of an amount of intake fluid dispensed to an individual. The fluid excretion unit may be configured to provide fluid excretion data, wherein the fluid excretion data is indicative of an amount of output fluid excreted by the individual. The hydration monitoring unit may be configured to receive the fluid intake data and the fluid excretion data and further configured to determine a hydration status of the individual based on a combination of the amount of intake fluid and the amount of output fluid.

An example of executable instructions for controlling a hydration monitoring system may include code for monitoring a hydration status based on a combination of intake and output fluid information. The intake fluid information may be indicative of an amount of fluid delivered to an individual and the output fluid information may be indicative of an amount of fluid expelled by the individual. In some examples, executable instructions may include instructions for providing an alarm based on the hydration status corresponding to one of a plurality of hydration states.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting of its scope, the disclosure will be described with additional specificity and detail through use of the accompanying drawings, in which:

FIG. 1 is a block diagram of an example hydration monitoring system;

FIG. 2 is another example of a hydration monitoring system;

FIG. 3 is an example chart showing changes in a user's hydration status over time;

FIGS. 4A and 4B are flowcharts illustrating example methods for monitoring hydration status;

FIG. 5 is a block diagram illustrating an example computing device that is arranged for hydration monitoring; and

FIG. 6 is a block diagram illustrating an example computer program product that is arranged to store instructions for hydration monitoring;

all arranged in accordance with at least some embodiments of the present disclosure.

### DETAILED DESCRIPTION

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are implicitly contemplated herein.

Real-time hydration monitoring of people and animals in a variety of settings may be desired. The desire to monitor a hydration status may help provide medical care to patients and animals and may also assist athletes by providing data on attaining maximum physical performance based on their hydration status and changes thereof during training sessions. Outside of the athletics realm, individuals in critical environments where heavy perspiration due to physical exertion may lead to fatigue and other maladies may include first responders, emergency personnel, and military personnel, for example. The hydration status of animals, such as working animals and animals under veterinary care, may also be of concern and monitoring desired. In these cases, it may be important for these individuals and animals to maintain hydration. Due to the lag between ingestion of a hydrating fluid and distribution of the fluid into the tissues, maintaining proper hydration may be difficult and the body's indicators may come too late for maintenance of optimal hydration. Thus, techniques for real-time monitoring of hydration status may be desired in a multitude of settings.

This disclosure is drawn, inter alia, to methods, systems, products, devices, and/or apparatus generally related to determining a hydration status of a user based on a combination of fluid intake and fluid output. The fluid intake may be based on an amount of fluid gained by an individual and may also be indicative of a composition of the fluid, such as electrolytes and nutrient contents for example. The fluid output value may be indicative of an amount of fluid lost by an individual and may further indicate the composition of the lost fluid. Based on the fluid intake and the fluid output, a hydration monitor may inform the user of a hydration status and whether the user should or should not consume (or be provided) fluids. Additionally, the composition of the provided fluid may be indicated and/or controlled by the hydration monitor. Alternatively or additionally, the hydration monitor may predict when a user should begin to hydrate based at least partially on a rate of change of the user's hydration status and the direction of the change being toward a dehydrated status.

The described techniques for monitoring hydration status may provide an array of benefits. For example, the monitoring of hydration status over time may allow users to maintain optimal hydration as the monitor may alert them when and how much fluid, either volume and/or composition, to consume. Long term data may also provide comprehensive understanding of an individual's normal hydration status and the affects that changes to the status have on the individual.

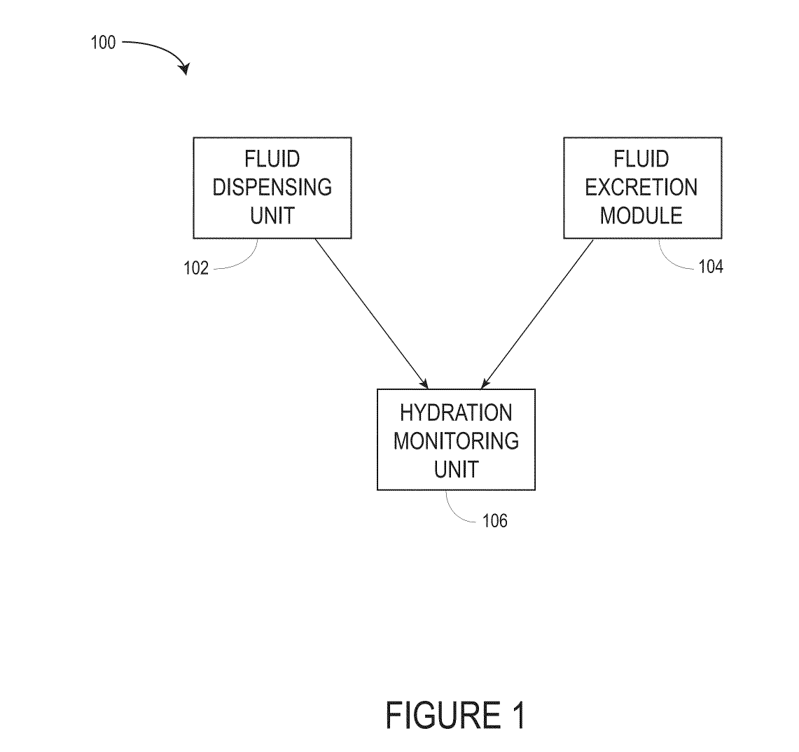

FIG. 1 is a block diagram of a hydration monitoring system 100 arranged in accordance with at least some embodiments described herein. FIG. 1 shows fluid excretion module 104, fluid dispensing unit 102, and hydration monitoring unit 106. The fluid dispensing unit 102 may be in communication with the hydration monitoring unit 106 using any of a variety of techniques, such as Bluetooth, Wi-Fi, radio communication or through a physical connection, for example. Similarly, the fluid excretion module 104 may also be in communication with the hydration monitoring unit 106 using any of the same techniques. Alternatively or additionally, the various components 102, 104, and 106 of the hydration monitoring system 100 may be in communication via the internet or a telephony system. The various components described in FIG. 1 are merely examples, and other variations, including eliminating components, combining components, and substituting components are all contemplated.

The fluid excretion module 104 may monitor fluid loss of a user, e.g., an athlete, a medical patient, an animal, and may provide data indicative of a volume of fluid loss to the hydration monitoring unit 106. For example, fluid output (fluid loss) due to an individual sweating may be monitored, measured, and the volume of the sweat loss per time may be provided to the hydration monitoring unit 106. The volume of lost fluid indicated may be dependent upon the type of monitor utilized, however. Other monitors may include measurements of water loss due to urination, breathing, and emesis for a few examples, which may be monitored by weight or volume. Alternatively or additionally, the fluid excretion module 104 may provide data indicative of a composition of the lost fluid. The composition may include the type and amount of electrolytes in the lost fluid, the type and amount of nutrients in the lost fluid, or combinations thereof.

The fluid dispensing unit 102 may monitor fluid dispensed to the user, which may be indicative of fluid intake by the user, and may also provide composition information of the fluid to the hydration monitoring unit 106. The data may indicate a volume of fluid consumed and/or a composition of the fluid consumed. The fluid dispensing unit 102 may be used with any type of vessel (e.g., a water bottle, a store-bought sports drink, or an intra-venous delivery system) and may include a meter to measure volume and/or a sensor to measure composition of the fluid. For example, volume may be measured using a turbine flow meter, a Woltmann flow meter, or an optical flow meter. To measure composition, for example, an ion sensitive field effect transistor may be used, which changes current according to ion concentration in the fluid.

Additionally, the fluid dispensing unit 102 may be able to control the volume and/or the composition of the dispensed fluid. For example, the fluid dispensing unit 102 may be able to control a volume of fluid dispensed when the composition of the fluid is pre-known. Alternatively, the fluid dispensing unit 102 may be able to control a mixture of water and electrolytes/nutrients so that a fluid of a desired composition is dispensed at a target volume. For example, a medical patient receiving fluid intra-venuously may require an additive, such as a nutrient, electrolyte or even medication added to the saline solution depending on analysis of fluid being output by the patient. The output fluid may be due to emesis, diarrhea, urination, sweating, or combinations thereof. The fluid dispensing unit 102, in this example, may control what type of additive and what amount to add to the saline solution.

The hydration monitoring unit 106 may receive the data indicative of the user's fluid intake and the fluid output. The hydration monitoring unit 106 may combine the fluid intake and the fluid output data and provide a hydration status to the user. For example, the hydration monitoring unit 106 may subtract the fluid output value from the fluid intake value and then add the resulting value to a resulting value from a previous calculation. The value may fall within a range of value indicating a specific hydration status. Optionally, the hydration monitoring unit 106 may provide control signals to the fluid dispensing unit 102 to control the volume and/or composition of the dispensed fluid. The control signals for controlling the volume and/or composition of the fluid may be based on the data regarding the output fluid.

Additionally, the hydration monitoring unit 106 may receive user-specific data that may impact the determination of the user's hydration status. User-specific data that may have an impact on the hydration status may include age, gender, body mass index (BMI), weight, and fitness level, all which may be input into the hydration monitoring system 100. Other parameters that may also affect a user's hydration status may be either manually input or automatically retrieved by the hydration monitoring system 100. Such parameters may be the heart rate of the user, which may be time-dependent to capture changes in exertion level, accelerometer data to show activity level, and information regarding the user's surrounding such as temperature, humidity, heat index, etc. The hydration monitoring unit 106 may evaluate all received parameters in addition to the fluid loss/gain parameters when determining a hydration status of the user.

The hydration monitoring unit 106 may determine a level of total body water turnover (TBWT), which may indicate a hydration status of the user. The TBWT may be determined from the difference of fluid input (based on the amount/composition of the fluid dispensed) less the fluid output. This may essentially be the balance of the two, which may be important to maintenance of optimal hydration, particularly in situations where there is high turnover, such as during exercise or certain medical conditions. The hydration monitoring unit 106 may also account for salt extrusion and other metabolic factors that may affect the hydration status of the user based on the composition of the fluid lost, when measured. Thus, by knowing the amount of fluid being consumed and the amount of water being excreted/eliminated from the user, the hydration monitoring system may monitor the TBWT of the user and provide a signal alerting the user of the determined hydration status.

The hydration monitoring unit 106 may provide an output to alert the user to the user's current hydration status. The alerts may be audible, visual and/or tactile (e.g., a vibration). The alerts may inform the user of being in one of a plurality of hydration statuses. The plurality of hydration statuses may include dehydrated, slightly dehydrated, optimal, slightly over-hydrated, and over-hydrated, for example. The hydration monitoring system 100 may alert the user when the user is in an undesirable hydration status, such as slightly dehydrated or dehydrated, for example, which should inform the user to consume fluid, such as water or a sports drink. The system may do the same for the over-hydrated statuses and an alert informing the user not to consume any fluid may be provided.

Dehydration, in general and as used herein, may imply the loss of water and salts essential for normal body function, for example. As the degree of dehydration may change, such as to slightly dehydrated, the amount of water and salts for essential normal body function may also change. In this light, slightly dehydrated, as used herein, may imply the loss of around 5% of the body's fluid. As water loss approaches 10% and then 15%, the degree of dehydration may increase from moderate to severe. In contrast, optimal hydration, in general and as used herein, may imply that fluid within and outside of cells within a body are in balance. Over-hydration, as used herein, may imply a condition in which a body contains too much water, which dilutes the sodium levels. Reduced sodium levels may result in a variety of health issues, including digestive problems, behavioral changes, brain damage, etc., and may be just as serious as becoming dehydrated. The relative hydration terms and their example meanings are used for illustrative purposes and should not be considered limiting to the present disclosure.

The range and boundaries of the hydration status levels may be influenced by the user-specific parameters, such as age, BMI, fitness level, and gender for example. Initially for a user, the levels and associated boundaries may conform to general medical guidelines based on statistics for an average person with similar user-specific parameters. Over time, however, the hydration monitoring unit 106 may adjust the ranges and boundaries based on gathered data for the specific user. For example, the user's level of fitness may affect the range and boundaries of the various hydration status levels. Changes to the hydration status levels may occur periodically or they may be continuously adjusted.

Optionally, the hydration monitoring unit 106 may control the volume and/or composition of the fluid being dispensed by the fluid dispensing unit 102. The hydration monitoring unit 106 may provide control signals to the fluid dispensing unit 102 for providing a pre-determined volume of fluid. The control signals may also dictate a composition of the fluid. Based on the fluid loss data (volume and composition), the hydration monitoring unit 106 may determine an amount and/or composition of fluid the user should consume (or be provided) to maintain the optimal hydration status or to improve the hydration status from a less desired hydration status to the optimal status.

An example hydration monitoring system 100 may include a fluid dispensing unit that includes a flow meter, a fluid excretion module, and a hydration monitoring unit. The fluid output monitor may be included on or coupled to a beverage container so that the volume and/or composition of the fluid being dispensed from the beverage container may be measured and measurement values provided to the hydration monitoring unit. The fluid excretion module may be attached to the body of the individual so that sweat loss may be measured. The sweat loss measurements may measure the volume and/or composition of the sweat excreting form the individual. The fluid excretion module, depending on type and placement, may also provide measurement of fluid loss from other bodily functions such as urination, emesis, etc. The value(s) of the fluid loss measurements may then be provided to the hydration monitoring unit. The hydration monitoring unit may be combined with either the fluid dispensing unit or the fluid excretion module or it may be a standalone component. For example, the hydration monitoring unit may be an application operating on a smartphone or mobile computer and the various components of the hydration monitoring system 100 may be communicating wirelessly.

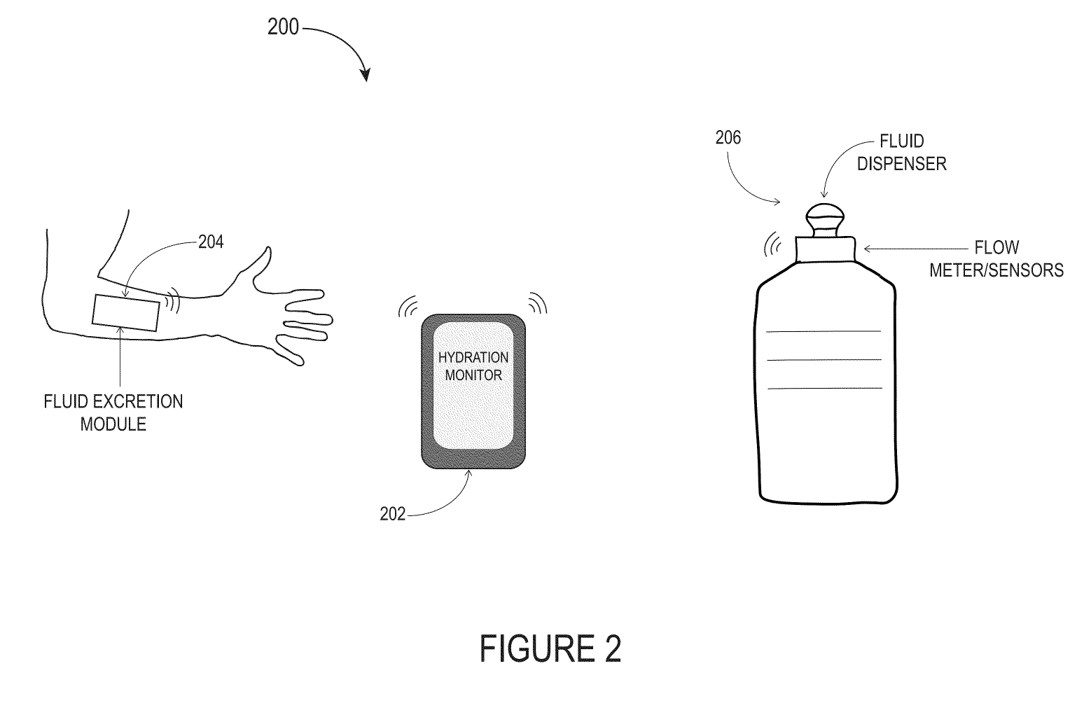

FIG. 2 is an example hydration monitoring system 200 arranged in accordance with at least some embodiments described herein. The hydration monitoring system 200 may include a hydration monitor 202, a fluid excretion module 204, and a fluid dispenser 206. The fluid dispenser 206 may be in wireless communication with the hydration monitor 202 via Bluetooth, Wi-Fi, or radio communication. The fluid excretion module 204 may similarly be in wireless communication with the hydration monitor 202. The components of the hydration monitoring system 200 may operate in concert to provide hydration status updates to a user. Additionally, the hydration monitoring system 200 may alert the user when and when not to consume fluids in order to maintain an optimal hydration status, based at least in part on various parameters associated with the user. The various components described in FIG. 2 are merely examples, and other variations, including eliminating components, combining components, and substituting components are all contemplated. For example, the hydration monitor 202 and the fluid dispenser 206 may be combined into a single component that both monitors fluid intake by the user and alerts the user of hydration status and prompts the user to consume fluids.

The fluid excretion module 204 may be similar to the fluid excretion module 102 of FIG. 1. The user may wear the fluid excretion module 204 to monitor loss of fluid through the user's skin, which may be due to perspiration for example. The fluid excretion module 204 may adhere to the skin of the user so to monitor fluid volume and/or content loss. The module may be disposable, which may allow the user to discard the module at the end of a hydration monitoring session, such as an athletic training cycle. A reusable module may be incorporated into a wearable device that a user may wear on their arm, wrist, or torso for extended periods of time. For example, a fluid excretion module 204 may be incorporated into a heart rate monitor, a smart watch, or an activity tracker. The reusable module may be worn by a user to monitor their hydration status throughout the day, or while exercising or participating in a physical activity.

The fluid excretion module 204 may further include components to measure other characteristics regarding the user. These other characteristics may include the user's heart rate, the user's body temperature, and an activity level of the user. The measurements may be made using accelerometers, temperature sensors, and pressure sensors. This data may also be provided to the hydration monitor 202. In example embodiments where the fluid excretion monitor 204 is incorporated into a heart rate monitor or activity tracker, the hosting device may be in communication with the hydration monitor 202 (or it may be the hydration monitor 202) and the various other physical-oriented data collected by the heart rate monitor/activity tracker may be also be communicated to the hydration monitor 202.

The fluid dispenser 206 may be attached to a drinking bottle, for example, and may measure volumes of fluid dispensed to the user. The fluid dispenser 206 may attach to standard water or sports drink bottles and may include flow meters and/or sensors for measuring the amount and/or electrolyte content of the fluid dispensed. The measured data may then be transmitted to the hydration monitor 202. Additionally, the fluid dispenser 206 may provide alerts to the user when an amount of fluid has been dispensed so the user does not overhydrate. For example, the hydration monitor 202, based on a current hydration status, may provide control information to the fluid dispenser 206 informing the dispenser how much fluid the user should ingest to ensure the user's hydration status remains in an optimal range.

The hydration monitor 202 may be a handheld computing device or a smartphone and may receive fluid loss and fluid dispensed data from the fluid excretion module 204 and the fluid dispenser 206, respectively. The hydration monitor 202 may combine the data to determine a hydration status of the user. The user may then be informed of their hydration status periodically or upon being prompted by the user. Other factors may also be considered by the hydration monitor 202 when determining a hydration status of the user. The other factors may be manually entered into by the user or may be automatically obtained by the hydration monitor 202. For example, characteristics of the user may be entered into the monitor by the user such as age, gender, height, weight, BMI, etc. Other factors such as ambient temperature, humidity, the user's heart rate, and the user's body temperature, for example, may be obtained by the hydration monitor through various means. The hydration monitor 202, for example, may obtain weather information via an internet connection and may receive heart rate and body temperature information from the fluid excretion module 204. The determination of the user's hydration status by the hydration monitor 202 may then be enhanced based on the additional information, e.g., user characteristics, weather, activity levels.

The hydration monitor 202 may inform the user of the user's current hydration status and may also provide alerts intended to prompt the user to consume fluid when the user's hydration status begins to decrease. The time-changing hydration status of the user may be continuously monitored by the hydration monitor 202 so that the monitor may predict changes to the user's hydration status. Based on the predictions, the hydration monitor 202 may warn the user to drink prior to the user entering an undesirable hydration status, such as under hydrated or dehydrated. Warning/alerts may be provided by audible, visual, or tactile alerts. The type and intensity of the alerts provided by the hydration monitor 202 may increase in volume and period to alert the user to hydrate if the user's hydration status is quickly approaching less desirable state, e.g., dehydrated.

When the hydration monitoring system 200 is being used during a training cycle, for example, the user may be able to start and stop monitoring process through the hydration monitor 202. In this user scenario, the user may first input their personal data used for determining the hydration status and may initiate the communication links between the various components 202, 204, and 206. Once the system is in communication, the user may then set a baseline hydration level from which the monitor may begin assessment. The user may then periodically check the monitor for their hydration status or wait to be alerted to their status and to intake fluids. The user may then terminate the monitoring session at the end of their training and save their data for further use or analysis.

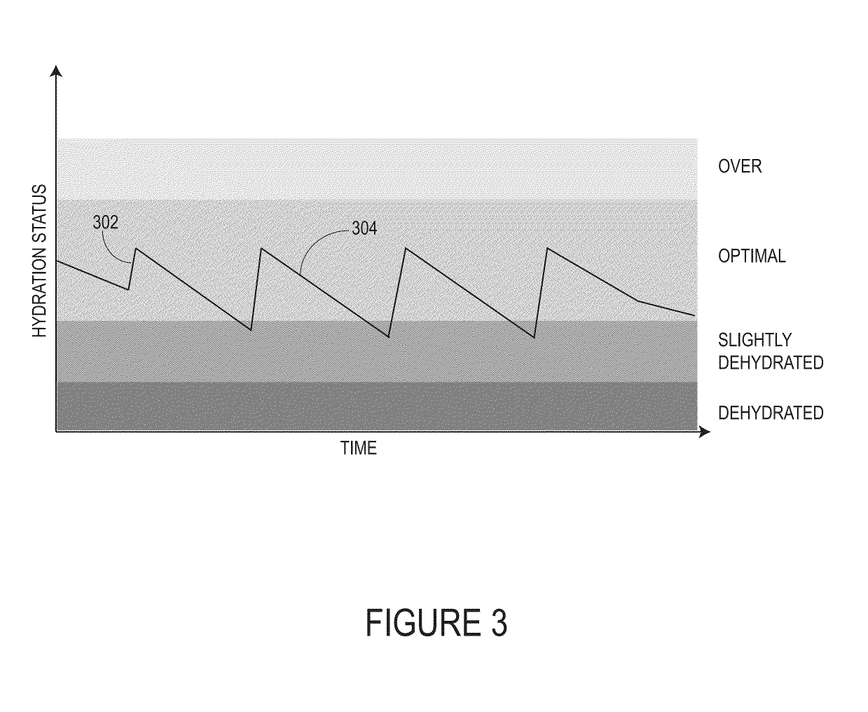

FIG. 3 is an example chart 300 showing changes in a user's hydration status over time. The chart 300 may show fluid loss (represented by the line 304) and fluid gained (represented by the line 302) by a user over a period of time. The chart 300 may be an example of the hydration monitoring system 100 and/or the hydration monitoring system 200 tracking input and output (loss) of fluid over time to determine when to provide an alert to a user to hydrate or not hydrate. Due to a lag between ingestion of a fluid and the absorption of the fluid into a user's tissues, the hydration monitoring system may provide pre-emptive alerts to the user when it predicts impending changes to the user's hydration status. In a case where the user may be experiencing heavy water loss (e.g., extreme physical exertion or a severe medical condition) the user may be informed more frequently or more aggressively when and how much to rehydrate. The trends may be determined by the hydration monitoring system in real time (e.g., over short time frames such as minutes, hours, or based on longer term data). For example, the hydration monitoring system may learn over time the individual's trends and lags between ingestion and hydration for a set of conditions (e.g., a particular exertion level as determined by accelerometer data) and determine hydration averages based on that data. Further, the input and output data may be augmented by the other data discussed above, such as the temperature, heart rate, and activity levels, for example. Furthermore, the hydration monitoring system may integrate seasonal or longer term trend information to further refine the predictive feature.

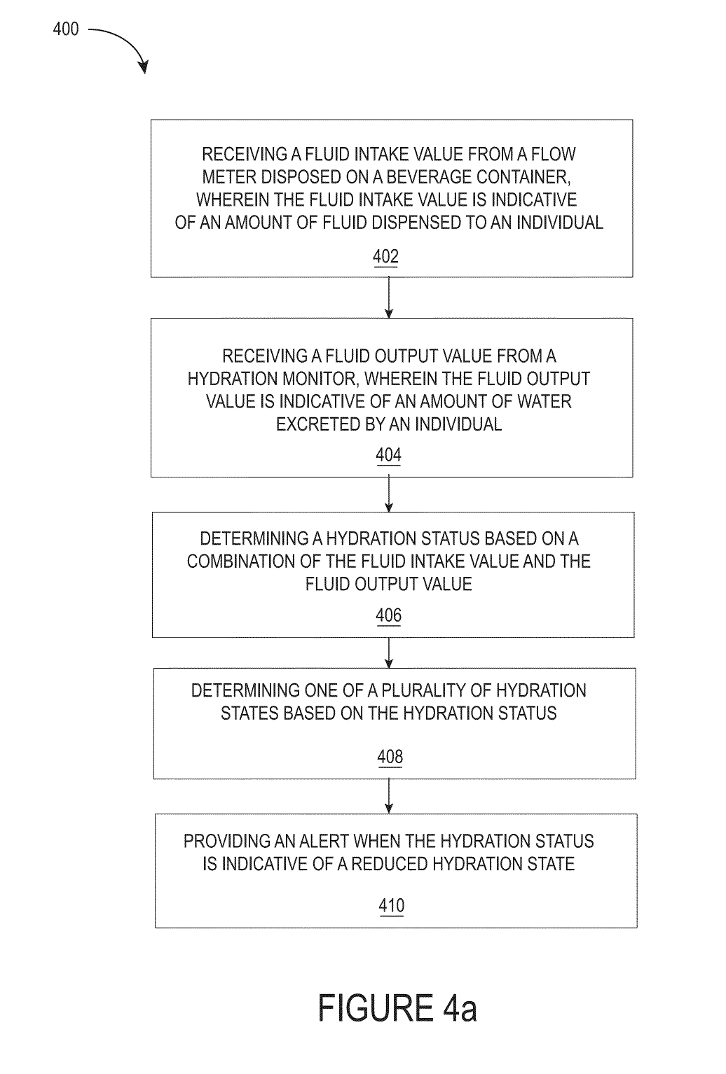

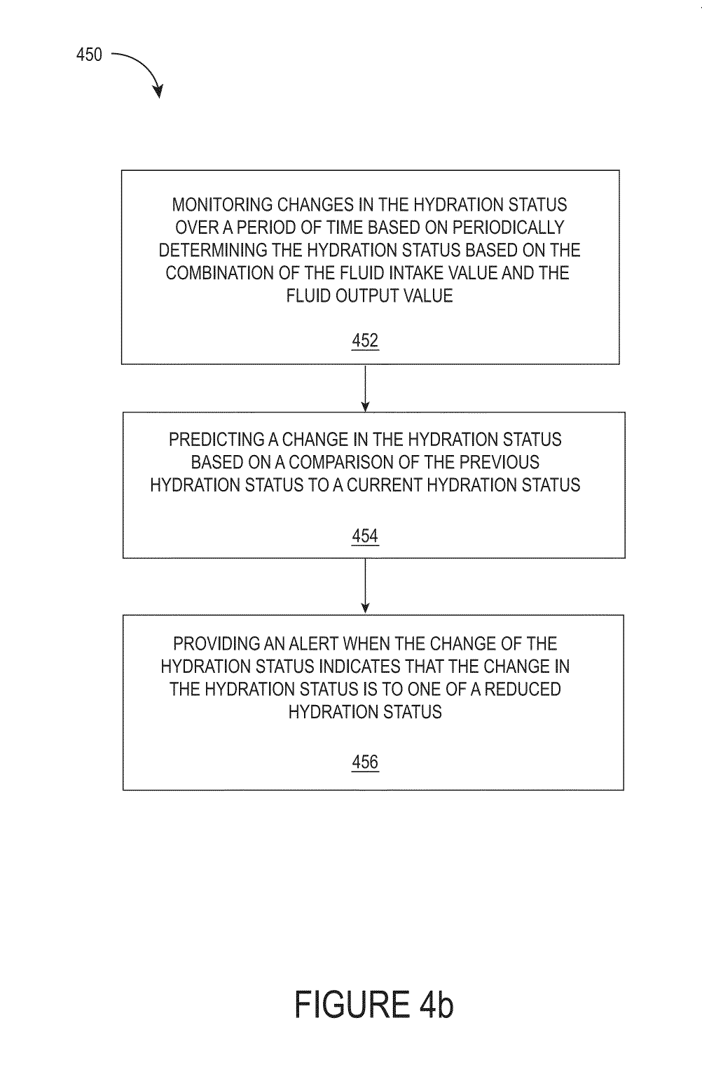

FIG. 4A is an example flowchart illustrating an example method 400 for monitoring hydration status in accordance with the present disclosure. An example method 400 may include one or more operations, functions or actions as illustrated by one or more of blocks 402, 404, 406, 408 and/or 410. The operations described in the blocks 402 through 410 may be performed in response to execution (such as by one or more processors described herein) of computer-executable instructions stored in a computer-readable medium, such as a computer-readable medium of a computing device or some other controller similarly configured.

An example process may begin with block 402, which recites “receiving a fluid intake value from a flow meter disposed on a beverage container, wherein the fluid intake value is indicative of an amount of fluid dispensed to an individual.” Block 402 may be followed by block 404, which recites “receiving a fluid output value by a fluid excretion module, wherein the fluid output value is indicative of an amount of fluid excreted by the individual.” Block 404 may be followed by block 406, which recites “determining a hydration status based on a combination of the fluid intake value and the fluid output value.” Block 406 may be followed by block 408, which recites “determining one of a plurality of hydration states based on the hydration status.” And block 408 may be followed by block 410, which recites “providing an alert when the hydration status is indicative of a reduced hydration state.”

Block 402 recites, “receiving a fluid intake value from a flow meter disposed on a beverage container, wherein the fluid intake value is indicative of an amount of fluid dispensed to an individual.” The flow meter may be part of a fluid dispensing unit, which may be in communication with a hydration monitoring unit, both of which may be components of a hydration monitoring system. The flow meter may measure a volume of fluid dispensed from the beverage container by an individual, for example. The fluid dispensing unit may be attached to or coupled to the beverage container in a manner that allows the flow meter to measure the volume of fluid dispensed from the beverage container by the individual. Additionally, the fluid dispensing unit may measure the electrolyte and/or nutrient composition of the fluid. The fluid dispensing unit may then provide the fluid intake value and/or composition to the hydration monitoring unit.

Block 404 recites, “receiving a fluid output value by a fluid excretion module, wherein the fluid output value is indicative of an amount of fluid excreted by the individual.” The fluid output value may be measured by a fluid excretion module, for example, and may indicate a volume of fluid loss per by the individual. The fluid excretion module may provide the fluid output value to the hydration monitoring unit. Additionally, the fluid excretion module may also provide information regarding salt and/or nutrient content of the lost fluid. The volume and/or composition data may then be provided to the hydration monitoring unit.

The fluid output value, e.g., volume and/or composition, of the fluid excreted by the individual may further include volumes and/or compositions of fluids excreted by the individual due to other bodily functions. For example, a flow and composition monitor may determine or estimate an amount of fluid lost through urination. The value indicating the loss of fluid through urination may also be provided to the hydration monitoring unit.

Block 406 recites, “determining a hydration status based on a combination of the fluid intake value and the fluid output value.” The hydration monitoring unit determines the hydration status of the individual by combining the fluid intake value and the fluid output value. For example, the hydration monitoring unit may subtract the fluid output value from the fluid intake value to produce a measure/value indicative of the individual's total body water turnover (TBWT). The value of the TBWT may indicate an overall hydration status of the individual.

Additionally, the hydration monitoring unit may further take into account characteristics specific to the individual and other data in determining the hydration status. For example, the hydration monitoring unit may take into account the loss (volume and type) of electrolyte measured in the excreted fluid. Further, the user-specific data, such as age, gender, fitness level, and heart rate may also affect the hydration status determination. Moreover, current weather patterns and any physical exertion the user is exhibiting may further affect the analysis. Thus, the hydration monitoring unit may include these various factors in determining the hydration status of the user.

Block 408 recites, “determining one of a plurality of hydration states based on the hydration status.” The hydration monitoring unit may determine a hydration state based on the hydration status. The value of the TBWT may fall within a range of one of a plurality of hydration states. For example, a high fluid output value in combination with a low fluid intake value may indicate a low measure of TBWT, which may indicate a poor hydration status, such as dehydrated or slightly dehydrated.

Block 410 recites, “providing an alert when the hydration status is indicative of a reduced hydration state.” The hydration monitoring unit may then indicate the individual's hydration state by providing an alert, which may be visual, audible, and/or tactile. For example, the hydration monitoring unit may blink a light emitting diode and produce a sequence of beeps to indicate a specific hydration state. Additionally or alternatively, if the hydration monitoring unit is included in a smartphone or a portable device, the hydration monitoring unit may produce a text-based alert along with an audible/visual/tactile alert to provide to the individual.

FIG. 4B is another example flowchart illustrating an example method 450 for monitoring hydration status in accordance with the present disclosure. An example method 450 may include one or more operations, functions or actions as illustrated by one or more of blocks 452, 454, and/or 456. The operations described in the blocks 452 through 456 may also be performed in response to execution (such as by one or more processors described herein) of computer-executable instructions stored in a computer-readable medium, such as a computer-readable medium of a computing device or some other controller similarly configured.

An example process may begin with block 452, which recites “monitoring changes in the hydration status over a period of time based on periodically determining the hydration status based on the combination of the fluid intake value and the fluid output value.” Block 452 may be followed by block 454, which recites “predicting a change in the hydration status based on a comparison of a previous hydration status to a current hydration status.” And block 454 may be followed by block 456, which recites “providing an alert when the change of the hydration status indicates that the change in the hydration status is to one of a reduced hydration status.”

Block 452 recites, “monitoring changes in the hydration status over a period of time based on periodically determining the hydration status based on the combination of the fluid intake value and the fluid output value.” The hydration monitoring unit may periodically receive the fluid intake and fluid output values from their respective components. At each interval, the hydration monitoring unit may alert the individual to the current hydration status based on the current values and/or on a combination of the current values and prior values. Since the hydration monitoring unit may accumulate hydration status data over a period of time, then hydration monitoring unit may at some point in time begin to predict trends in the individual's hydration state based on the gathered data and the user-specific data. Due to the time-based monitoring of the individual's hydration status, the hydration monitoring unit may be able to generate a time-based hydration status profile for that current monitoring event, such as shown in FIG. 3.

Block 454 recites, “predicting a change in the hydration status based on a comparison of a previous hydration status to a current hydration status.” Then, based on the monitoring of the time-dependent hydration status, the hydration monitoring unit may be able to predict future changes in the individual's hydration status and a rate of change of that status. For example, an athlete in a training session being monitored by the hydration monitoring unit may go long periods of time between taking in fluids and over time, the hydration monitoring unit may be able to predict future changes to the athlete's hydration status based on a determined rate of change and data regarding exertion levels of the athlete. By comparing previous and current hydration statuses, the hydration monitoring unit may determine how the status is changing and how rapidly.

Block 456 recites, “providing an alert when the change of the hydration status indicates that the change in the hydration status is to one of a reduced hydration status.” Based on the predicted change to the individual's hydration status, the hydration monitoring unit may provide an alert when the individual should take in some fluid to increase the hydration state. By alerting the individual to drink, for example, before the hydration status shows slightly dehydrated or dehydrated, the individual may avoid the lag time between ingestion and tissue absorption of the fluid to maintain an optimal hydration and performance level.

The blocks included in the described example methods are for illustration purposes. In some embodiments, the blocks may be performed in a different order. In some other embodiments, various blocks may be eliminated. In still other embodiments, various blocks may be divided into additional blocks, supplemented with other blocks, or combined together into fewer blocks. Other variations of these specific blocks are contemplated, including changes in the order of the blocks, changes in the content of the blocks being split or combined into other blocks, etc. In some examples, determining a hydration status may be further based on physiological parameters of the individual, such as age, gender, body mass index, level of fitness, etc.

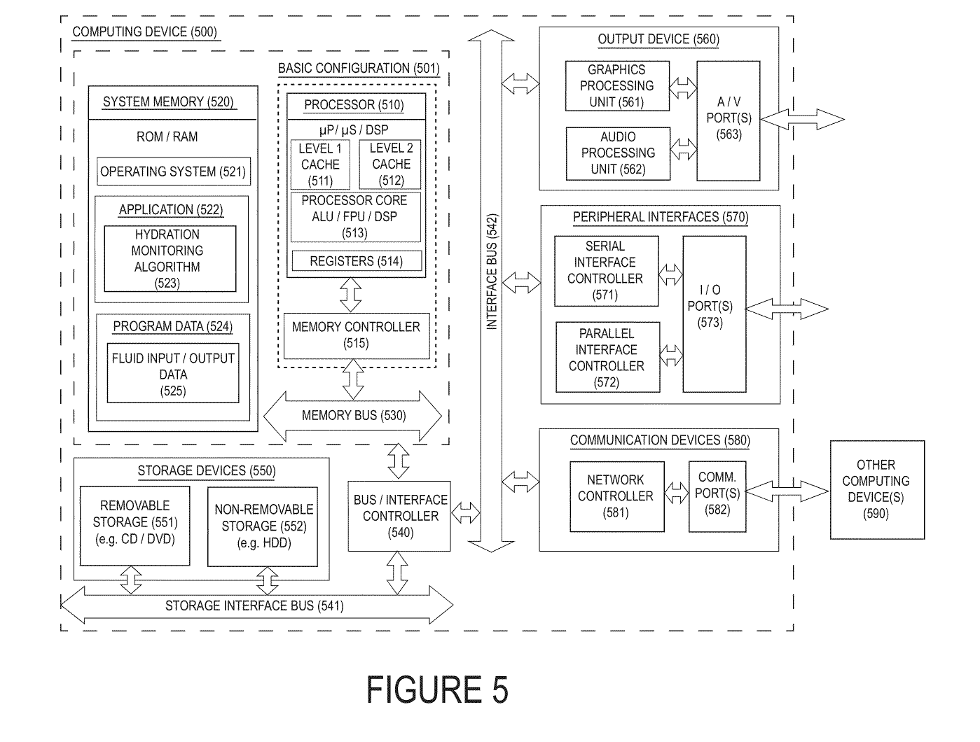

FIG. 5 is a block diagram illustrating an example computing device 500 that is arranged for hydration monitoring in accordance with the present disclosure. In a very basic configuration 501, computing device 500 typically includes one or more processors 510 and system memory 520. A memory bus 530 may be used for communicating between the processor 510 and the system memory 520.

Depending on the desired configuration, processor 510 may be of any type including but not limited to a microprocessor (μP), a microcontroller (μC), a digital signal processor (DSP), or any combination thereof. Processor 510 may include one more levels of caching, such as a level one cache 511 and a level two cache 512, a processor core 513, and registers 514. An example processor core 513 may include an arithmetic logic unit (ALU), a floating point unit (FPU), a digital signal processing core (DSP Core), or any combination thereof. An example memory controller 515 may also be used with the processor 510, or in some implementations the memory controller 515 may be an internal part of the processor 510.

Depending on the desired configuration, the system memory 520 may be of any type including but not limited to volatile memory (such as RAM), non-volatile memory (such as ROM, flash memory, etc.) or any combination thereof. System memory 520 may include an operating system 521, one or more applications 522, and program data 524. Application 522 may include a hydration monitoring procedure 523 that is arranged to monitor the hydration status of a user as described herein. Program data 524 may include individual's characteristics and fluid input and fluid output data, and/or other information useful for the implementation of hydration monitoring. In some embodiments, application 522 may be arranged to operate with program data 524 on an operating system 521 such that any of the procedures described herein may be performed. This described basic configuration is illustrated in FIG. 5 by those components within dashed line of the basic configuration 501.

Computing device 500 may have additional features or functionality, and additional interfaces to facilitate communications between the basic configuration 501 and any required devices and interfaces. For example, a bus/interface controller 540 may be used to facilitate communications between the basic configuration 501 and one or more storage devices 550 via a storage interface bus 541. The storage devices 550 may be removable storage devices 551, non-removable storage devices 552, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 520, removable storage 551 and non-removable storage 552 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 500. Any such computer storage media may be part of computing device 500.

Computing device 500 may also include an interface bus 542 for facilitating communication from various interface devices (e.g., output interfaces, peripheral interfaces, and communication interfaces) to the basic configuration 501 via the bus/interface controller 540. Example output devices 560 include a graphics processing unit 561 and an audio processing unit 562, which may be configured to communicate to various external devices such as a display or speakers via one or more A/V ports 563. Example peripheral interfaces 570 include a serial interface controller 571 or a parallel interface controller 572, which may be configured to communicate with external devices such as input devices (e.g., keyboard, mouse, pen, voice input device, touch input device, etc.) or other peripheral devices (e.g., printer, scanner, etc.) via one or more I/O ports 573. An example communication device 580 includes a network controller 581, which may be arranged to facilitate communications with one or more other computing devices 590 over a network communication link via one or more communication ports 582.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 500 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 400 may also be implemented as a personal computer including both laptop computer and non-laptop computer configurations.

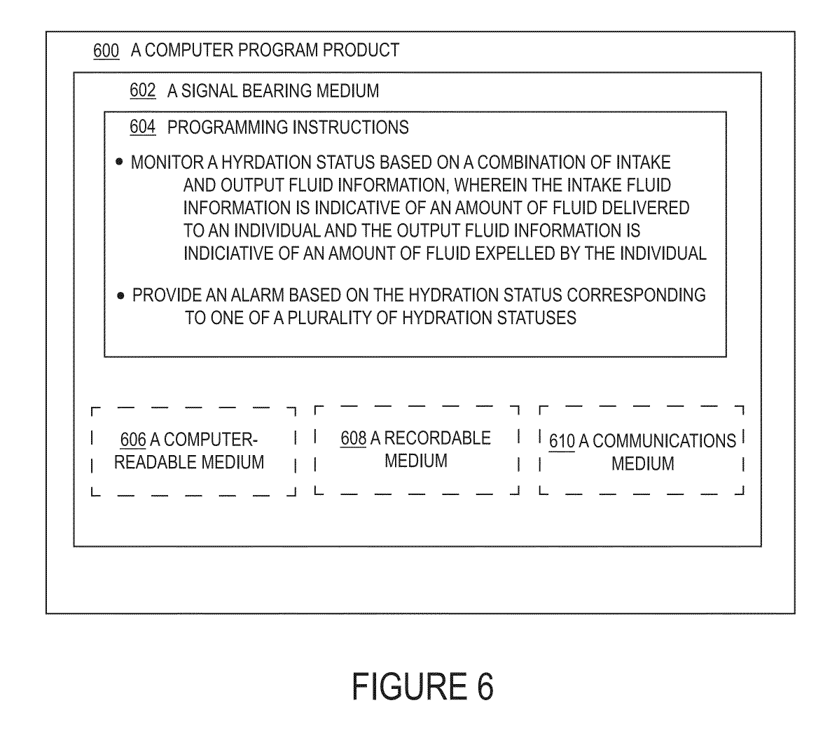

FIG. 6 is a block diagram illustrating an example computer program product 600 that is arranged to store instructions for hydration monitoring in accordance with the present disclosure. The signal bearing medium 602 which may be implemented as or include a computer-readable medium 606, a computer recordable medium 608, a computer communications medium 610, or combinations thereof, stores programming instructions 604 that may configure the processing unit to perform all or some of the processes previously described. These instructions may include, for example, one or more executable instructions for monitoring a hydration status based on a combination of intake and output fluid information, wherein the intake fluid information is indicative of an amount of fluid delivered to an individual and the output fluid information is indicative of an amount of fluid expelled by the individual, and for providing an alarm based on the hydration status corresponding to one of a plurality of hydration states.

In some examples, a system for determining a hydration status comprises a fluid dispensing unit configured to provide fluid intake data, wherein the fluid intake data is indicative of an amount of intake fluid dispensed to an individual, a fluid excretion module configured to provide fluid excretion data, wherein the fluid excretion data is indicative of an amount of output fluid excreted by the individual; and a hydration monitoring unit, configured to receive the fluid intake data and the fluid excretion data and configured to determine a hydration status of the individual based on a combination of the amount of intake fluid and the amount of output fluid.

In some examples, a hydration monitoring unit may be further configured to predict a change in hydration status based on a plurality of hydration status determinations over a span of time, and, based on the change in hydration status, and provide an alert when the change in hydration status indicates changes toward a low level of hydration. In some examples, an alert may include a visual alert (for example, illumination of one or more light emitting diodes, a text display, and the like), an audible alert (such as a tone, buzzing, synthesized speech, and the like), haptic alert (such as a perceptible vibration), other alert, or some combination thereof. In some examples, a hydration monitoring unit may be a portable electronic device carried by an individual, and may have additional functionality. In some examples, a hydration monitoring unit may further comprise (or be provided by) a portable computer, portable phone, wristwatch, eyewear, a helmet, and the like. In some examples, a hydration monitoring unit may further comprise a strap to secure the hydration monitoring unit to a body part.

In some examples, a system comprises at least one of a fluid dispensing unit, a fluid excretion module, and a hydration monitoring unit. In some examples, a system includes at least two of a fluid dispensing unit, a fluid excretion module, and a hydration monitoring unit in communication with each other, for example using wireless communication.

In some examples, a fluid dispensing unit comprises, attaches to, or otherwise cooperates with a fluid container. A fluid container may comprise, for example, a standard beverage container or a reusable fluid container. In some examples, a fluid dispensing unit may replace or fit over a standard cap at the dispensing portion of the fluid container. A fluid dispensing unit may comprise a threaded inlet portion configured to receive the threaded neck portion of a beverage container, and receive fluid dispensed from the fluid container. A fluid dispensing unit may further comprise an outlet portion configured to dispense fluid to an individual. An example fluid dispensing unit may comprise a flow meter configured to determine the amount (e.g. weight and/or volume) of fluid flowing as it is dispensed from a fluid container. In some examples, a fluid dispensing unit comprises a turbine flow meter, a Woltmann flow meter, an optical flow meter, or other flow meter. In some examples, a fluid dispensing unit may include a weight sensor and be configured to determine the fluid dispensed from a weight change in the fluid in the fluid container. In some examples, a fluid dispensing unit may comprise one or more sensors, such as an ion-sensitive field-effect transistor (ISFET) sensor, to measure the composition of the beverage being dispensed, for example to measure electrolyte, salt (e.g. sodium chloride), other anion and/or cation, and/or sugar concentration in the beverage. In some examples, electrolyte balance and/or energy balance (e.g. nutrient intake compared with monitored activity and/or metabolic rate) may be determined.

In some examples, a fluid dispensing unit may be configured to dispense one or more predetermined volumes (or units) of fluid. The fluid dispensing unit may then be configured to count the number of units dispensed, allowing the volume to be determined from the number of units and the unit volume.

In some examples, a fluid dispensing unit may be configured to dispense a beverage of known composition. For example, the dispensing device may be constructed so as to fit a standard beverage container that is pre-filled with a known drink, such as a particular sports drink. Determination of a volume of fluid dispensed also allows an amount and type of electrolytes and nutrients being consumed by the individual to be determined. In some examples, a fluid dispensing unit, or other electronic device in communication with the fluid dispensing unit (such as the hydration monitoring unit), may comprise a bar code reader and be configured to retrieve nutritional or other information relating to the fluid from a product identifying bar code.

In some examples, a fluid dispensing unit may be configured to control the amount and type of nutrient and/or electrolyte being dispensed. In some examples, an individual may add electrolyte and/or nutrients to a fluid, and record the additions, for example using in input interface of a hydration monitoring unit.

In some examples, a fluid dispensing unit comprises a user interface, such as one or more of buttons, lamps (such as LEDs), audible alerts, a display, and the like. A fluid dispensing unit may be configured to provide an alert to the user when it is recommended that the user should take a drink. An alert may comprise a visual alert, an audible alarm, other human-perceptible signal such as a vibration, other alert, or some combination thereof. A display, if present, may also provide other information to the user, such as the time, activity, an overall hydration status (e.g. of the individual, or other subject), or other data relating to exercise. In some examples, physiological data such as hydration may be displayed relating to the individual of an electronic device, which may include, attach to, or otherwise be in communication with a fluid dispensing device. In some examples, an individual may be an assistant, trainer, coach, doctor or other medical professional, dietician, other professional care-giver, or similar, and physiological data, alerts, and the like may be displayed or otherwise conveyed to the user relating to one or more subjects in the care of, or otherwise associated with, the individual. In some examples, a fluid dispensing unit is configured to provide an alert to an individual in response to a signal from a hydration monitoring unit. In some examples, an individual may, for example, depress a button when fluid is dispensed from the fluid container but not used for intake by the individual (for example when pouring fluid over a head or other body part).

A fluid dispensing unit may be configured to communicate with a hydration monitoring unit. In some examples, the communication may comprise a wireless communication, such as Bluetooth, Wi-Fi, or radio communication. A fluid dispensing unit may be configured to communicate an amount (e.g. weight and/or volume), and optionally composition data (such as electrolyte and/or nutrient content), of fluid dispensed to an individual, for example to a hydration monitoring unit and/or other device(s). A fluid dispensing unit may be configured to receive hydration information from a hydration monitoring unit relating to the hydration status of the person. Hydration information may comprise one or more of plasma load, tissue hydration, sweat rate, other physiological data, or some combination thereof. The amount of fluid dispensed may be assumed to be the same as the fluid intake by the individual, or in some examples a correction factor may be included, for example a predetermined correction factor, or a correction factor provided by an individual.

A fluid excretion module may include a perspiration sensor, which may be configured to be supported by an individual's body. A fluid excretion module, comprising one or more sensors, may be configured to measure or estimate fluid excretion, such as perspiration, and optionally urine, water vapor in breath, and the like. In some examples, a system may be configured to estimate an amount of fluid excretion (for example as a volume and/or weight of fluid lost), for example by estimating perspiration using data from sensors such as an activity sensor, heart rate sensor, ambient temperature sensor, body temperature sensor, humidity sensor, body weight measuring device (scales), acoustic sensor (e.g. to estimate urination amount, respiration volumes, and the like), respiration sensor (e.g. to estimate water vapor loss), and the like. In some examples, an electrolyte composition of excreted fluids may be estimated or determined, for example by electrical conductivity analysis of perspiration or urine, colorimetric analysis of urine, and/or chemical analysis of any excreted fluid. In some examples, a system may determine an amount of salt and/or nutrient lost by the individual, for example during a period of activity, and use this information to control the dispensation of a similar or otherwise related amount to the individual from a fluid dispensing unit. In some examples, a system may comprise one or more sensors or transducers, such as an accelerometer (for example, one or more accelerometers, such as one or more 3-axis sensing accelerometers, e.g. for activity determination), a temperature monitor (e.g. an ambient temperature sensor, core body temperature sensor, skin temperature sensor, or some combination thereof), a heart rate monitor, a humidity sensor, a respiration sensor, or other physiological or ambient condition sensor to hydration determination and estimates thereof. In some examples, a fluid excretion module may include, communicate with, or otherwise be configured to receive data from a weight and/or volume scale, for example for measurement or estimation of elimination weights and/or volumes. In some example, a fluid excretion module may include, communicate with, or otherwise be configured to receive data from a weight scale, for example a weight scale configured to determine a body weight of an individual. In some examples, a fluid excretion module may include a colorimeter, for example configured to determine a color of a fluid, such as an eliminated fluid. In some examples, fluid color data may be used in the estimation of hydration state, electrolyte balance, and the like. In some examples, eliminated fluid weight may be estimated from a weight change of a fluid absorption material configured to be placed proximate at least a portion of an individual's body. In some examples, the individual may be a baby or other infant. In some examples, a fluid-dispensing container may be configured for use with a baby or other non-adult human, an animal, or otherwise as desired.

In some examples, a system is configured for human use. In some examples, a system is configured to use with an animal, such as a mammal (e.g. a dog, cat, rodent, and the like), bird, reptile, and the like. For example, in an animal (e.g. canine) example, fluid intake may be determined using a weight sensor disposed underneath an animal (e.g. dog) water bowl. Animal (e.g. canine) fluid excretion may be estimated from ambient temperature, a pant sensor, measurement or estimation of weight and/or volume of elimination, and the like. Animal (e.g. canine) hydration data may be transmitted to a portable electronic device in possession of the animal owner.

The present disclosure is not to be limited in terms of the particular examples described in this application, which are intended as illustrations of various aspects. Many modifications and examples can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and examples are intended to fall within the scope of the appended claims. The present disclosure includes the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular examples only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

While the foregoing detailed description has set forth various examples of the devices and/or processes via the use of block diagrams, flowcharts, and/or examples, such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one example, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the examples disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. For example, if a user determines that speed and accuracy are paramount, the user may opt for a mainly hardware and/or firmware vehicle; if flexibility is paramount, the user may opt for a mainly software implementation; or, yet again alternatively, the user may opt for some combination of hardware, software, and/or firmware.

In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

What is claimed is:

1. A method of determining a hydration status of an individual, the method comprising:
receiving a fluid intake value from a flow meter disposed on a beverage container, wherein the fluid intake value is indicative of an amount of fluid dispensed to an individual;
receiving a fluid output value from a fluid excretion module, wherein the fluid output value is indicative of an amount of fluid excreted by the individual; and
determining a hydration status based on a combination of the fluid intake value and the fluid output value.

2. The method of claim 1, further comprising:
determining one of a plurality of hydration states based on the hydration status.

3. The method of claim 2, wherein the plurality of hydration states include over, optimal, slightly dehydrated, and dehydrated.

4. The method of claim 1, further comprising:
monitoring changes in the hydration status over a period of time by periodically determining the hydration status based on the combination of the fluid intake value and the fluid output value.

5. The method of claim 1, further comprising:
providing an alert when the hydration status is indicative of a reduced hydration state.

6. The method of claim 1, further comprising:
predicting a change in the hydration status based on a comparison of a previous hydration status to a current hydration status; and
providing an alert when the change of the hydration status indicates that the change in the hydration status is to one of a reduced hydration status.

7. The method of claim 1, wherein receiving the fluid intake value comprises wirelessly receiving the fluid intake value from the flow meter disposed on the beverage container.

8. The method of claim 1, wherein determining a hydration status based on a combination of the fluid intake value and the fluid output value is performed by a hydration monitoring unit.

9. The method of claim 1, wherein the fluid excretion module is worn by the individual.

10. The method of claim 1, wherein the hydration status includes total body water turnover, intake/excretion balance, electrolyte balance, and combinations thereof.

11. The method of claim 1, wherein the fluid output value includes an estimation of urination output.

12. A system for determining a hydration status, the system comprising:
a fluid dispenser unit configured to provide fluid intake data, wherein the fluid intake data is indicative of an amount of intake fluid dispensed to an individual;
a fluid excretion module configured to provide fluid excretion data, wherein the fluid excretion data is indicative of an amount of output fluid excreted by the individual; and
a hydration monitor module, configured to receive the fluid intake data and the fluid excretion data and configured to determine a hydration status of the individual based on a combination of the amount of intake fluid and the amount of output fluid.

13. The system of claim 12, wherein the hydration monitor module is further configured to predict a change in hydration status based on a plurality of hydration status determinations over a span of time, and, based on the change in hydration status, provide an alert when the change in hydration status indicates changes toward a low level of hydration.

14. The system of claim 12, wherein the hydration monitor module is further configured to predict a change in hydration status based on a plurality of hydration status determinations over a span of time, and, based on the change in hydration status, provide an alert when the change in hydration status indicates changes toward a high level of hydration.

15. The system of claim 12, wherein the fluid excretion module is further configured to provide composition data for the output fluid, wherein the composition data comprises an electrolyte level of the output fluid.

16. The system of claim 12, wherein the hydration status may correspond to one of over hydrated, slightly over hydrated, optimally hydrated, slightly dehydrated, and dehydrated.

17. The system of claim 16, wherein the hydration monitor module is configured to provide an alert when the hydration status is slightly dehydrated.

18. The system of claim 16, wherein the hydration monitor module is configured to provide an alert when the hydration status is over hydrated.

19. The system of claim 12, wherein the fluid dispenser unit is attached to a fluid container and the fluid dispenser unit is configured to determine the amount of intake fluid dispensed to the individual from the fluid container.

20. The system of claim 12, wherein the fluid excretion module is located on skin of the individual and is configured to determine the amount of output fluid excreted by the individual.

21. The system of claim 12, wherein the hydration monitor module is a portable electronic device in wireless communication with the fluid dispenser unit and the fluid excretion module.

22. At least one non-transitory computer-readable medium encoded with executable instructions configured to cause at least one processing unit to perform actions, the instructions comprising instructions to:
monitor a hydration status based on a combination of intake and output fluid information, wherein the intake fluid information is indicative of an amount of fluid delivered to an individual and the output fluid information is indicative of an amount of fluid expelled by the individual; and
provide an alarm based on the hydration status corresponding to one of a plurality of hydration states.

23. The non-transitory, computer-readable medium of claim 22, wherein the alarm is provided when the hydration status corresponds to an overhydrated hydration state.

24. The non-transitory, computer-readable medium of claim 22, wherein the alarm is provided when the hydration status corresponds to a dehydrated hydration state.

25. The non-transitory, computer-readable medium of claim 22, wherein monitor a hydration status based on a combination of intake and output fluid information comprises further instructions to:
determine a volume of intake fluid from a flow meter disposed on a fluid dispenser delivering the fluid to the individual;
determine a volume of output fluid from a hydration monitor monitoring the amount of fluid expelled by the individual; and
determine the hydration state based on dynamic changes between the volume of intake fluid and the volume of output fluid.

26. The non-transitory, computer-readable medium of claim 22, wherein the intake and output fluid information comprises volume and composition.
